(19) United States
(12) Patent Application Publication
(10) Pub. No.: US 2011/0098782 A1
(43) Pub. Date: Apr. 28, 2011

(54) LEAD EXTENSION HAVING CONNECTOR CONFIGURED TO RECEIVE TWO LEADS

(75) Inventors: John E. Kast, Hugo, MN (US); James A. Zimmerman, Biaine, MN (US); Craig S. Pilarski, Ham Lake, MN (US); William C. Phillips, Brooklyn Park, MN (US); Thomas I. Miller, Blaine, MN (US); Mary L. Boatwright, Andover, MN (US)

(73) Assignee: Medtronic ,Inc., Minneapolis, MN (US)

(21) Appl. No.: 12/992,945

(22) PCT Filed: May 29, 2009
(86) PCT No.: PCT/US2009/045575

§ 371 Date: Nov. 16, 2010

Related U.S. Application Data
(60) Provisional application No. 61/058,286, filed on Jun. 3, 2008.
(60) Provisional application No. 61/175,478, filed on May 5, 2009.

(51) Int. Cl. A61N 1/36; A61N 1/05

(52) U.S. Cl. 607/46; 607/116

(57) ABSTRACT

A lead extension includes a proximal portion having first and second contacts, and further includes a connector having a body. The body of the connector houses first and second lead receptacles. The first lead receptacle has an internal contact electrically coupled to the first proximal contact. The second lead receptacle has an internal contact electrically coupled to the second proximal contact.

### FIELD

The present disclosure relates to implantable medical devices; more particularly to medical leads capable of delivering electrical signals to two discrete anatomical locations, such as a left and a right occipital nerve.

### BACKGROUND

Headaches, such as migraines, and occipital neuralgia are often incapacitating and may lead to significant consumption of drugs to treat the symptoms. However, a rather large number of people are unresponsive to drug treatment, leaving them to wait out the episode or to resort to coping mechanisms. For refractive occipital neuralgia, nerve ablation or separation may effectively treat the pain.

Occipital nerve stimulation may serve as an alternative for treatment of migraines or occipital neuralgia. For example, a dual channel implantable electrical generator may be implanted subcutaneously in a patient. A distal portion of first and second leads may be implanted in proximity to a left and right occipital nerve such that one or more electrode of the leads are in electrical communication with the occipital nerves. The proximal portions of the leads may then be connected to the signal generator such that electrical signals can be delivered from the signal generator to the electrodes to apply therapeutic signals to the occipital nerves Alternatively, two single channel implantable electrical generators may be employed, where the first lead is connected to one signal generator and the second lead is connected to the second signal generator. In either case, the lead is typically tunneled subcutaneously from site of implantation of the signal generator to the occipital nerve or around the base of the skull. Such tunneling can be time consuming and is invasive.

### BRIEF SUMMARY

The present disclosure, among other things, describes leads, systems and methods for applying electrical signals to occipital nerves using a lead extension having two distal connectors, with a single proximal leg. By using such lead extensions, only one tunneling procedure is needed to tunnel a proximal portion of a lead between a location near the occipital nerves and the implantation site of the electrical signal generator. Such lead extensions and procedures may reduce surgery time and invasiveness associated with occipital nerve stimulation.

The connector regions of the lead extensions may occupy small volumes, e.g. less than 10 cubic centimeters, to allow for implantation in areas of the body that do not readily accommodate large implanted objects, such as the back of a patient's neck, near the base of the skull. Accordingly, the extensions may be well suited for use in occipital nerve stimulation therapies.

In an embodiment, a method for applying electrical signals to a left occipital nerve and a right occipital nerve of a subject are described. The method includes applying a first electrical signal to the left occipital nerve via an electrode of a first lead, and applying a second electrical signal to the right occipital nerve via a second electrode of a second lead. Applying the first and second electrical signal includes (i) generating the first electrical signal from an electrical signal generator implanted in the subject and transmitting the signal to the electrode of the first lead via a lead extension operably coupled to the first lead, and (ii) generating the second electrical signal from the electrical signal generator and transmitting the signal to the electrode of the second lead via the lead extension. The lead extension is operably coupled to the second lead. The first and second signals are the same or different. It will be understood that a signal may be transmitted between the electrode of the first lead and the electrode of the second lead via the tissue such that the signal is applied to the left or right occipital nerve. The lead extension may include a proximal portion that has first and second contacts and may include a distal connector portion. The distal connector portion has a body housing (i) a first lead receptacle having an internal contact electrically coupled to the first proximal contact, and (ii) a second lead receptacle having an internal contact electrically coupled to the second proximal contact.

In various embodiments, a lead extension is described. The extension includes a proximal portion having first and second contacts, and further includes a connector having a body. The body, which may be free or arms, extensions or the like, of the connector houses first and second lead receptacles. The first lead receptacle has an internal contact electrically coupled to the first proximal contact. The second lead receptacle has an internal contact electrically coupled to the second proximal contact.

The leads, extensions, signal generators, systems and methods described herein provide one or more advantages over prior leads, extensions, signal generators, systems and methods. Such advantages will be readily understood from the following detailed description when read in conjunction with the accompanying drawings.

### BRIEF DESCRIPTION OF THE DRAWINGS

FIG. 1 is a schematic side view of an implantable system including a signal generator, lead extension and leads.

FIGS. 2A-B are schematic diagrams showing leads and distal portions of lead extensions implanted in a subjects and positioned to apply electrical signals to left and right occipital nerves.

FIG. 3A is a schematic side view of a representative lead extension and associated leads.

FIG. 3B is a schematic side view of an embodiment of the connector region of the extension depicted in FIG. 3A, showing lead receptacles and showing conductors running through the connector region.

FIGS. 3C-E are schematic cross-sections of alternative embodiments of the proximal portion of the extension shown in FIG. 3A taken through line 3c-3c.

FIGS. 4A is a schematic side view of a lead extension and associated leads.

FIG. 4B is a schematic cross-section of a connector of FIG. 4A, showing some representative components, including receptacles for receiving leads.

FIGS. 5A-C are schematic drawings of lines running in a plane, showing embodiments of angles at which the receptacles of a connector portion of an extension may enter a body of the connector.

FIGS. 6-7 are schematic side views of representative extensions and associated leads.

FIGS. 8A-E and FIGS. 9A-F are schematic side views of representative lead extensions and associated leads.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”.

“Exemplary” or “representative” is used herein in the sense of “for example” or “for purposes of illustration”, and not in a limiting sense.

The present disclosure, among other things, describes leads, systems and methods for applying electrical stimulation signals to occipital nerves using a lead extension having two distal connectors, with a single proximal leg. By using such lead extensions, only one tunneling procedure is needed to tunnel a proximal portion of a lead between a location near the occipital nerves and the implantation site of the electrical signal generator. Such lead extensions and procedures may reduce surgery time and invasiveness associated with occipital nerve stimulation.

Nearly any implantable medical device or system employing leads may be used in conjunction with the leads, extensions or adaptors described herein. Representative examples of such implantable medical devices include hearing implants, cochlear implants; sensing or monitoring devices; signal generators such as cardiac pacemakers or defibrillators, neurostimulators (such as spinal cord stimulators, brain or deep brain stimulators, peripheral nerve stimulators, vagal nerve stimulators, occipital nerve stimulators, subcutaneous stimulators, etc.), gastric stimulators; or the like. For purposes of occipital nerve stimulation, electrical signal generators such as Medtronic, Inc.'s Restore® or Synergy® series of implantable neurostimulators may be employed.

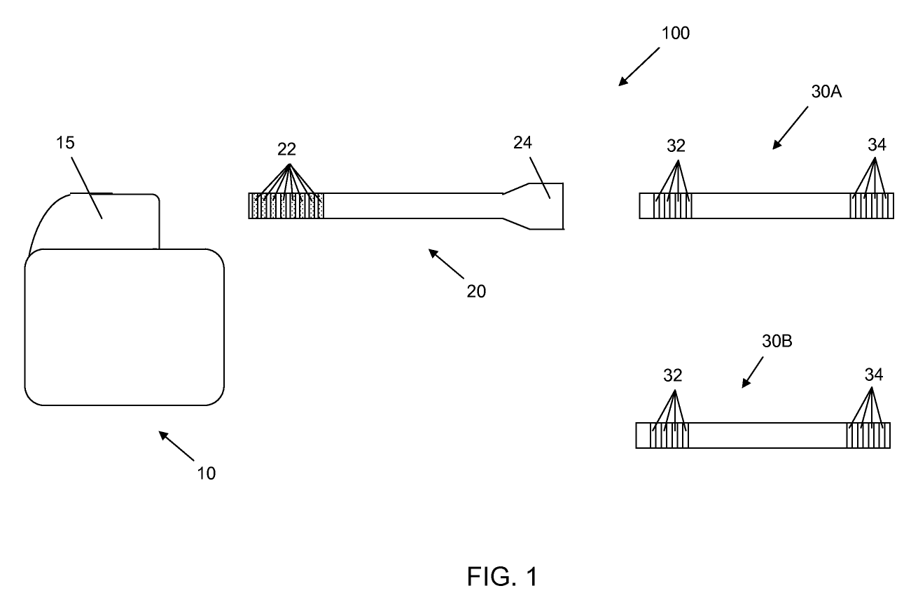

Referring to FIG. 1, a schematic side view of a representative electrical signal generator system 100 is shown. In the depicted system 100, the electrical signal generator 10 includes a connector header 15 configured to receive a proximal portion of lead extension 20. The proximal portion of lead extension 20 contains a plurality of electrical contacts 22 that are electrically coupled to internal contacts (not shown) at distal connector 24 of lead extension 20. The connector header 15 of the signal generator 10 contains internal contacts (not shown) and is configured to receive the proximal portion of the lead extension 20 such that the internal contacts of the connector header 15 may be electrically coupled to the contacts 22 of the lead extension 20 when the lead extension 20 in inserted into the header 15.

The system depicted in FIG. 1 further includes a leads 30A, 30B. The depicted leads 30A, 30B have a proximal portion that includes a plurality of contacts 32 and a distal portion that includes a plurality of electrodes 34. Each of the electrodes 34 of a lead may be electrically coupled to a discrete contact 32. The distal connector 24 of the lead extension 20 has two lead receptacles (not shown) configured to receive the proximal portion of the leads 30A, 30B such that the contacts 32 of the lead 30 may be electrically coupled to the internal contacts (not shown) of the connector 24 of the extension 20. Accordingly, a signal generated by the signal generator 10 may be transmitted to a patient by an electrode 34 of lead 30A, 30B when lead is connected to extension 20 and extension 20 is connected to signal generator 10.

Any number of leads 30A, 30B or extensions 20 may be coupled to signal generator 10. Typically, one or two leads 30A, 30B or extensions 20 are coupled to signal generator 10. While lead 20 is depicted as having four electrodes 34, it will be understood that lead 30 may include any number of electrodes 34, e.g. one, two, three, four, five, six, seven, eight, sixteen, thirty-two, or sixty-four. Corresponding changes in the number of contacts 32 in lead 30, contacts 22 and internal contacts in connector 24 of lead extension, or internal contacts in connector 15 of signal generator 10 may be required or desired.

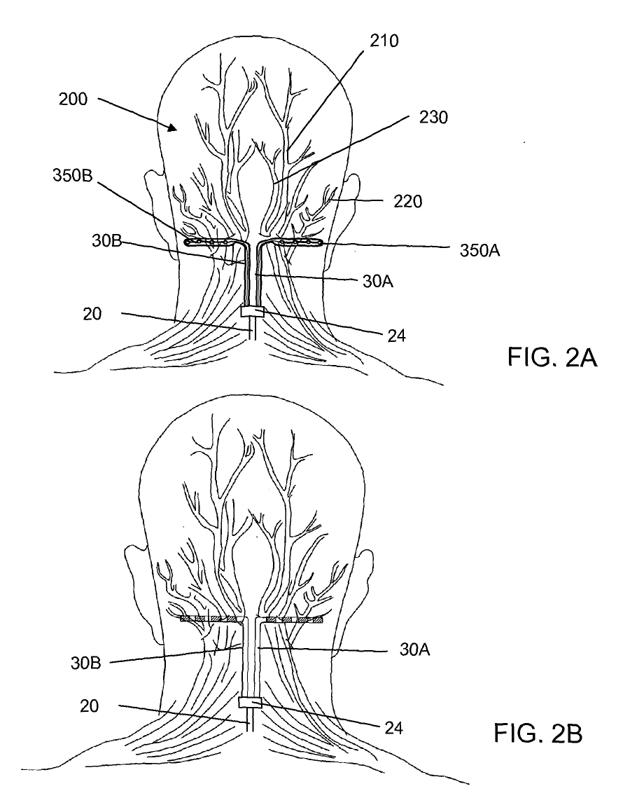

Referring to FIGS. 2A-B, representative lead extensions 20 and leads 30A, 30B are shown implanted in a patient to provide bilateral therapy to left and right occipital nerves 200. As used herein, occipital nerve 200 includes the greater occipital nerve 210, the lesser occipital nerve 220 and the third occipital nerve 230. The greater and lesser occipital nerves are spinal nerves arising between the second and third cervical vertebrae (not shown). The third occipital nerve arises between the third and fourth cervical vertebrae. The portion of the occipital nerve 200 to which an electrical signal is to be applied may vary depending on the disease to be treated and associated symptoms or the stimulation parameters to be applied. In various embodiments, the lead distal portions 350A, 350B (see FIG. 2A) that contain electrodes are placed to allow bilateral application of electrical signals to the occipital nerve 200 at a level of about C1 to about C2 or at a level in proximity to the base of the skull. The position of the electrode(s) may vary. It will be understood that the electrode need not, and in various embodiments preferably does not, contact the nerve to apply the signal to the nerve. It will be further understood that a signal may be applied to any suitable portion of an occipital nerve, whether at a trunk, branch, or the like. In various embodiments, one or more electrodes are placed between about 1 cm and about 8 cm from the midline to effectively provide an electrical signal to the occipital nerve 200.

As shown in FIG. 2A, a leads 30A, 30B may include a paddle shaped distal portion 350A. 350B containing electrodes. Such paddle shaped leads are often referred to as surgical leads. Examples of surgical leads that may be modified to form leads as described herein include Medtronic Inc.'s Resume, SymMix, On-Point, or Specify series of leads. Surgical leads typically contain electrodes that are exposed through one face of the paddle, providing directional stimulation. As shown in FIG. 2B, the leads 30A, 30B may include a distal portion that is generally cylindrically shaped. Such leads are often referred to as percutaneous leads. Examples of percutaneous leads that may be modified to form leads as described herein include Medtronic Inc.'s Quad Plus, Pisces Quad, Pisces Quad Compact, or 1×8 SubCompact, 1×8 Compact, and 1×8 Standard leads. Such percutaneous leads typically contain ring electrodes that apply an electrical stimulation signal to tissue in all directions around the ring. Accordingly, the amplitude of the signal (and thus the energy required from the signal generator) applied may be greater with percutaneous leads than surgical leads for occipital nerve therapies.

As shown in FIGS. 2A-B, a lead extension 20 capable of connecting to two leads 30A, 30B may have a single proximal leg that allows for only one tunneling procedure to the signal generator implant site (not shown), which may be any suitable location, such as the buttocks, abdomen, pectoral region, flank, pericostal region, or the like. The extension 20 includes a connector 24 configured to receive proximal portions of leads 30A, 30B to operably couple the extension 20 to the leads 30A, 30B.

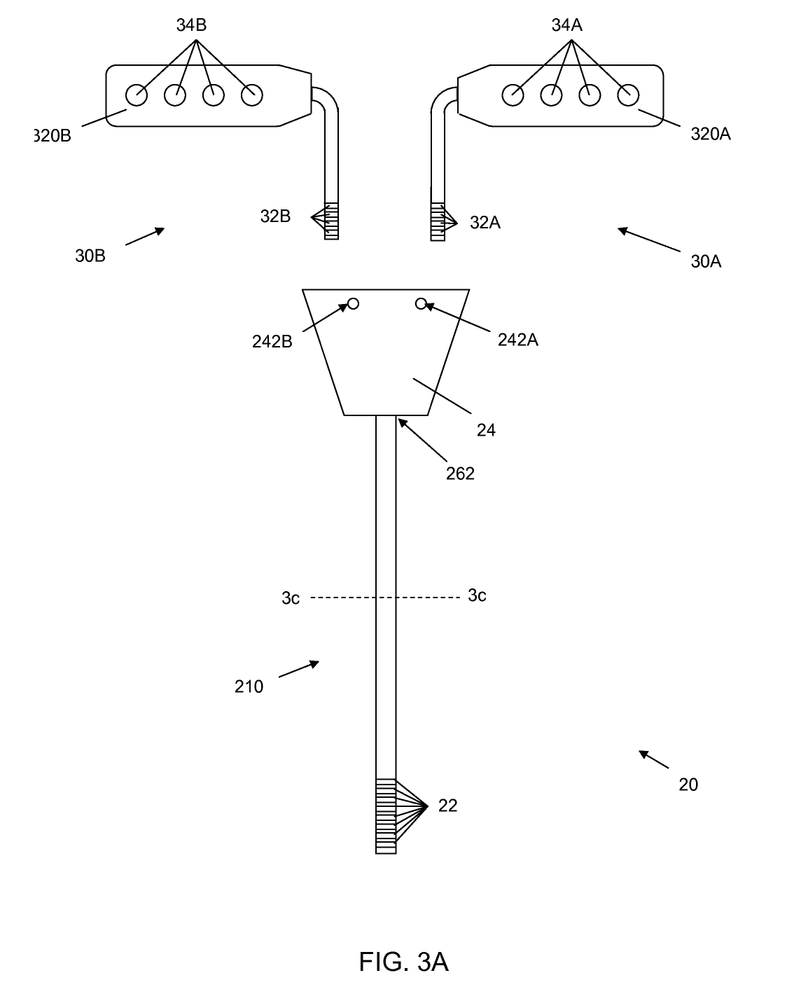

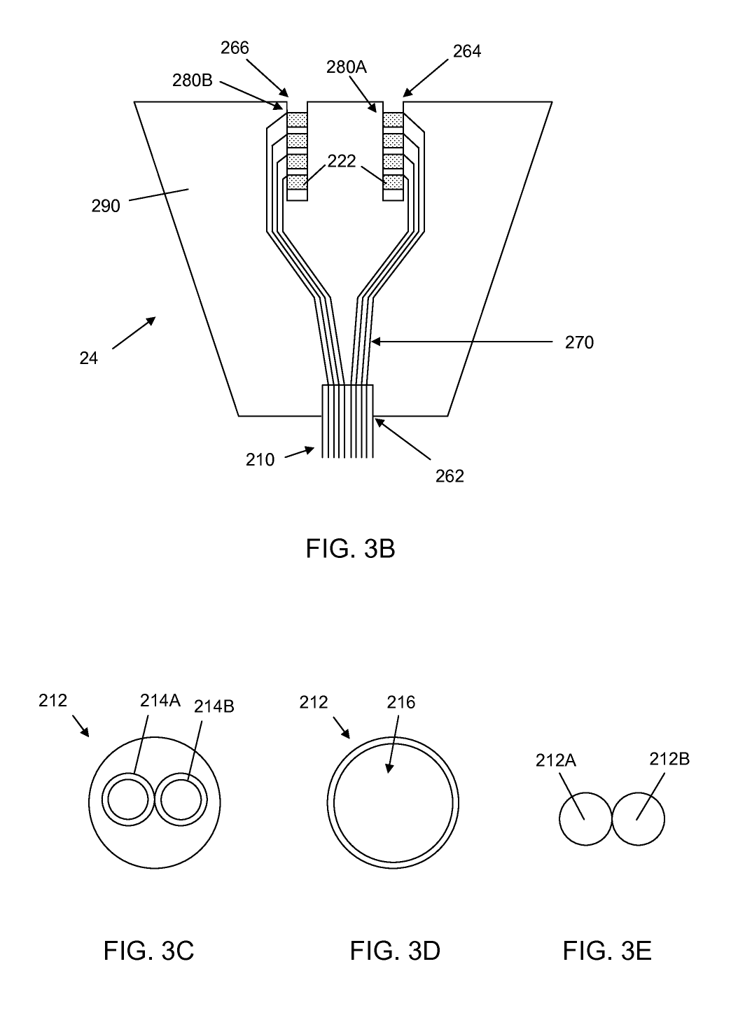

Various embodiments of lead extensions or system configurations are described below with reference to FIGS. 3-9. However, it will be understood that any lead extension and lead configuration may be employed to apply an electrical signal to an occipital nerve; e.g., as described above with regard to FIGS. 2A-B. It will be further understood that, while the lead extensions and system configurations described below may be useful for applying electrical signals to occipital nerves, they may be employed to apply electrical signals to other tissues of a subject or may be used to record signals from tissue of a subject.

Referring now to FIG. 3A, a schematic side view of a representative lead extension 20 and leads 30A, 30B is shown. The leads 30A, 30B include distal portions 320A, 320B that contain electrodes 34A, 34B. The electrodes 34A, 34B are electrically coupled to contacts 32A, 32B via conductors that run within lead 30A, 30B from the contacts 32A, 32B to the electrodes 34A, 34B.

The extension 20 depicted in FIG. 3A includes a proximal portion 210 that includes a plurality of contacts 22 for electrically coupling to an electrical signal generator. The extension 20 further includes a connector region 20 having lead receptacles (see, e.g., FIG. 3B) configured to receive proximal portions of the leads 30A, 30B. The receptacles contain internal contacts (see, e.g., FIG. 3B) configured to electrically couple with the contacts 32A, 32B of the leads 30A, 30B when the leads are received by the receptacles. The connector region 24 includes set screws 242A, 242B or other suitable screw-less mechanism for retaining the leads 30A, 30B in the receptacles. The connector region 24 may be of any suitable size and shape. In various embodiments, the connector region 24 has a volume of less than about 10 cubic centimeters, less than about 5 cubic centimeters, or less than about 2 cubic centimeters. Such small volumes may be desirable for implanting the connector region 24 in an area of a patient that cannot readily accommodate larger volumes, such as areas in the back of the neck where the connector region 24 may be implanted for occipital nerve therapies.

Referring now to FIG. 3B, a schematic cross sectional view of an embodiment of a connector region and a portion of the proximal portion of the lead extension of FIG. 3A is shown. In the depicted embodiment, a set of conductors 270 exit from the proximal portion 210 of the extension. The conductors 270 are independently coupled to internal connectors 222 of lead receptacles 280A, 280B. The internal contacts 222 are positioned and configured to form an electrical connection with contacts of a proximal portion of a lead, when the lead is inserted into the receptacle 280A, 280B. The receptacles 280A, 280B extend into the body 290 of the connector 24 from openings 264, 266 of formed in the body 290. The body 290 of the connector 240 may be formed of any suitable material, such as a suitable polymeric material. In various embodiments, the body 290 is overmolded over receptacles 280A, 280B, conductors 270 and a portion of the proximal portion 210 of the extension, which is shown entering the connector 24 at an entry region 262. Of course, any other suitable process may be used to form the connector region 24 of the extension.

Referring now to FIG. 3C-E, which is a cross section of the proximal portion 210 of the extension 20 depicted in FIG. 3A taken along line 3c-3c, showing representative configurations. As shown in FIG. 3C, the proximal portion of the extension includes a body 212. The body 212 may include two lumens or tubes 214A, 214B (or any number of tubes or lumens, e.g. one for each conductor) through which or around which conductors (not shown) may run to connect proximal contacts with internal contacts of the lead receptacles. Of course, the lumens or tubes 214A, 214B may be solid and the conductors can run in separate tracks along the length of the proximal portion of the extension until connecting with the internal contacts of the receptacles. Alternatively, as shown in FIG. 3D, the extension body 212 in the proximal portion may include a single lumen 216 or solid core (not shown) and the conductors (not shown) may run in a single track along the along the length of the proximal portion of the lead. Alternatively as shown in FIG. 3E, the proximal portion may include two (or more) attached bodies 212A, 212B through which separate channels of conductors (not shown) run. Of course, the lead body of the proximal portion of extension body may be configured in any other suitable manner.

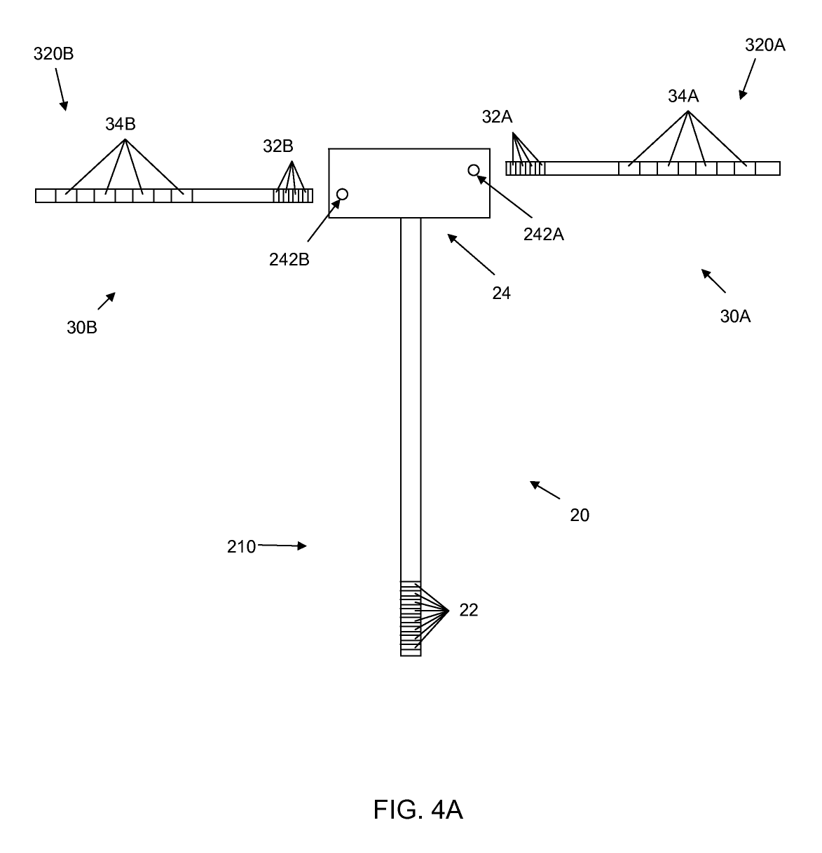

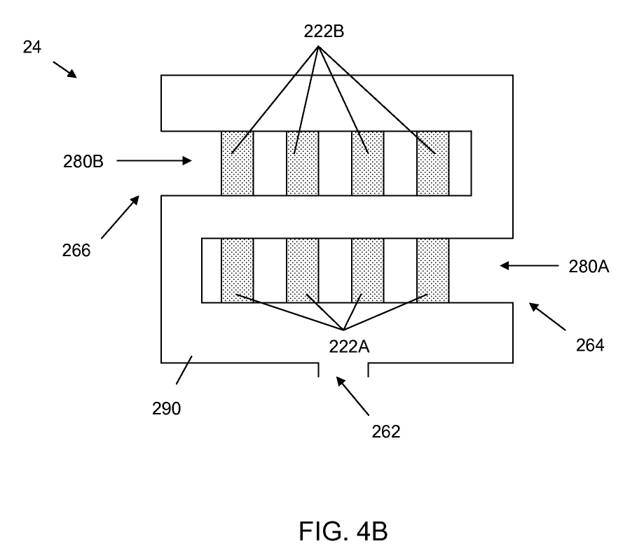

Referring now to FIG. 4A, a schematic drawing of an embodiment of a lead extension 20 and associated leads 30A, 30B is shown. The extension 20 has a distal connector 24 and a proximal portion 210 extending proximally from the connector 24. The proximal portion 210 of the extension 20 includes contacts 22 for electrical coupling the extension 20 to a signal generator or other suitable medical device. The distal portion of extension 20 includes a connector 24 containing two lead receptacles (see, e.g., FIG. 4B) having internal contacts for coupling to contacts 32A, 32B of leads 30A, 30B. The connector 24 may be of any suitable size and shape. In various embodiments, the connector 24 has a volume of less than about 10 cubic centimeters, less than about 5 cubic centimeters, or less than about 2 cubic centimeters. Set screws 242A, 242B may be used to secure leads 30A, 30B in receptacles. Of course, any other suitable mechanism for securing leads 30A, 30B in receptacles may be employed. In the embodiment depicted in FIG. 4, the lead receptacles (not shown) are generally perpendicular to the angle of entry of the proximal portion 210 into connector 24.

Leads 30A, 30B include proximal portions containing contacts 32A, 32B and distal portions 320A, 320B containing electrodes 34A, 34B. By employing an extension having a connector 24 as described herein, separate leads 30A, 30B standard introducer tools, such as needle introducers with lumens, may be used to position distal portion 320A, 320B of leads 30A, 30B. For bifurcating leads alternative methods for introducing distal portions may be desired.

Referring now to FIG. 4B, a schematic cross-section of an embodiment of the connector portion 24 of the lead extension 20 of FIG. 4A is shown. The connector 24 includes first 280A and second 280B lead receptacles. The receptacles 280A, 280B include openings on opposing ends of connector 24 for inserting leads in to the receptacles 280A, 280B and include internal contacts 222A, 222B for electrically coupling to contacts of leads when inserted into the receptacles 280A, 280B. In the depicted embodiment, the receptacles 280A, 280B extend into the body 290 of the receptacle 24 in a substantially parallel manner along a plane defined by the geometric centers (which may be axial centers) of the opening 264 of the first receptacle 280A, the opening 266 of the second receptacle 280B, and the entry region 262 of the proximal portion of the extension into the connector 24. The first 280A and second 280B each extend into the body 290 along this plane substantially perpendicular to a line in the plane extending through the geometric center of the entry region 262. Of course, the receptacles may extend into the body 290 at any suitable angle.

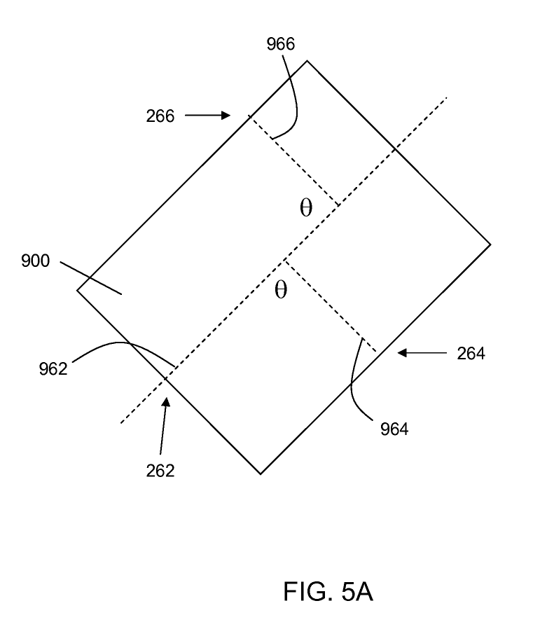

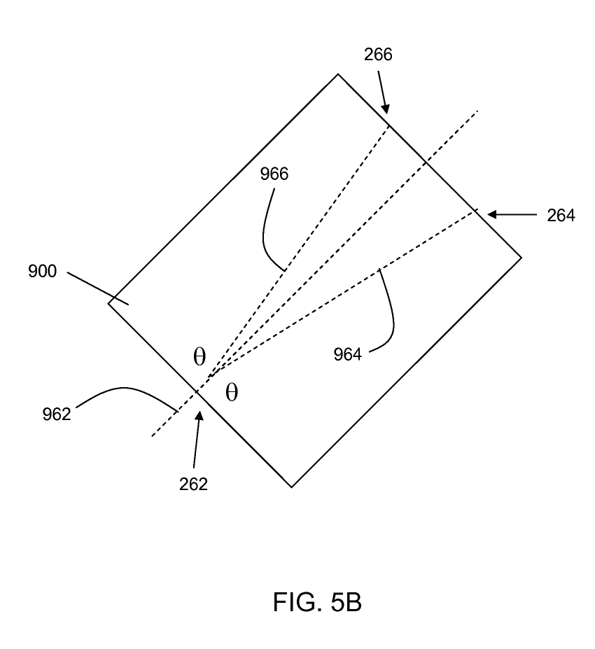

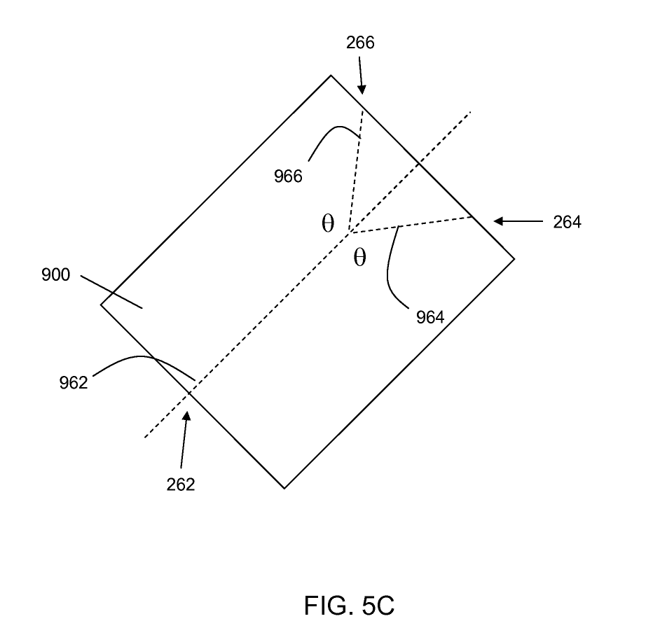

For example and with reference to FIGS. 5A-C, representative configurations are shown where receptacles enter the body of a connector at various angles are shown. In FIGS. 5A-C, a plane 900 is shown. The plane 900 is defined by the geometric centers of the entry region 262 where the proximal portion of the extension enters the connector, the first opening 264 formed by the body that defines an opening of the first receptacle, and the second opening 266 formed by the body that defines an opening of the second receptacle. Several lines 962, 964, 964 are shown running in the plane. Line 962 represents a line running through the geometric center of the entry point 262, which in some embodiments is the axial center of the proximal portion of the extension as it enters the connector. Lines 964 and 966 represent a line running through the axial center of the first and second lead receptacles. In various embodiments, lines 962 and 964 or lines 962 and 966 intersect at angles (indicated by “θ”) of between about 90 degrees and about 180 degrees. In some embodiments, the angles are between about 110 degrees and about 160 degrees.

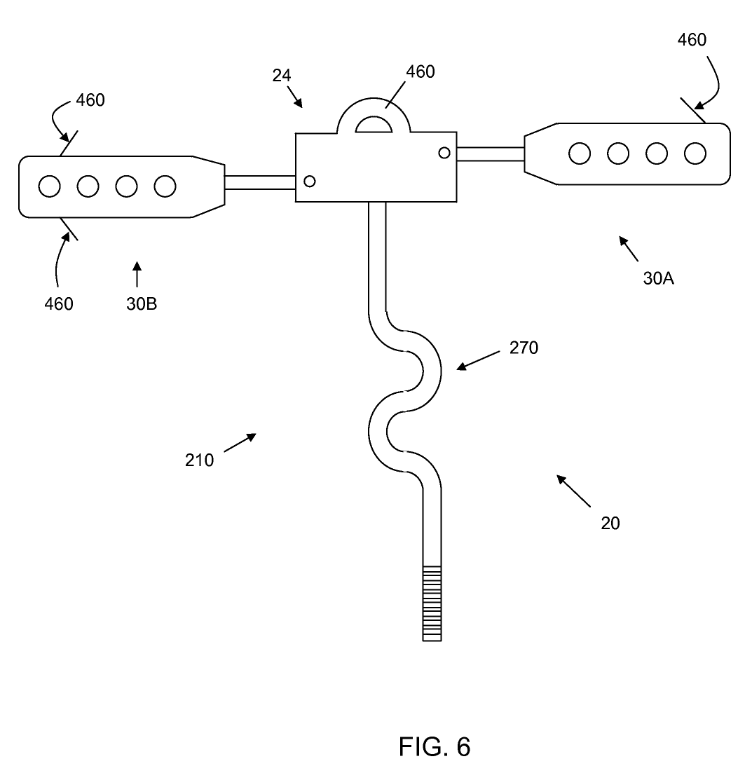

Referring now to FIG. 6, a lead 30A, 30B or extension 20 may include one or more anchors 460 for facilitating retention of the lead or extension to tissue in which it is implanted. In FIG. 6, the anchors 460 are depicted as suture holes or tines, but the anchors may take any suitable form. In various embodiments, an anchor 460 is attached to the connector region 440 of an extension 20. As used herein, “attached”, as it relates to an anchor and connector region of an extension, means to affix the anchor to the connector. The anchor is affixed or attached to the connector region well in advance of implantation, such as during the manufacture of the extension. In various embodiments, the anchor is permanently attached to the connector region.

For application of therapies to an occipital nerve, where proximal portion 210 is tunneled through the neck region of a subject, it may be desirable to securely anchor connector 24 to tissue of the subject to prevent movement of the lead (due to strains and stressed on the proximal portion 210) from causing movement of leads 30A, 30B connected to the connector 24. In addition, it may be desirable for proximal portion 210 to contain a strain relief feature to allow for stretching and movement of the neck to prevent or inhibit stress or strain at the proximal portion 210 from transferring to the leads 30A, 30B via the connector 24. For example, proximal portion 210 may include a sigma shaped portion 270, may be looped (not shown), or may be extensible. One or more anchors 460 may be attached to leads 30A, 30B, such as at the distal portions containing electrodes as depicted.

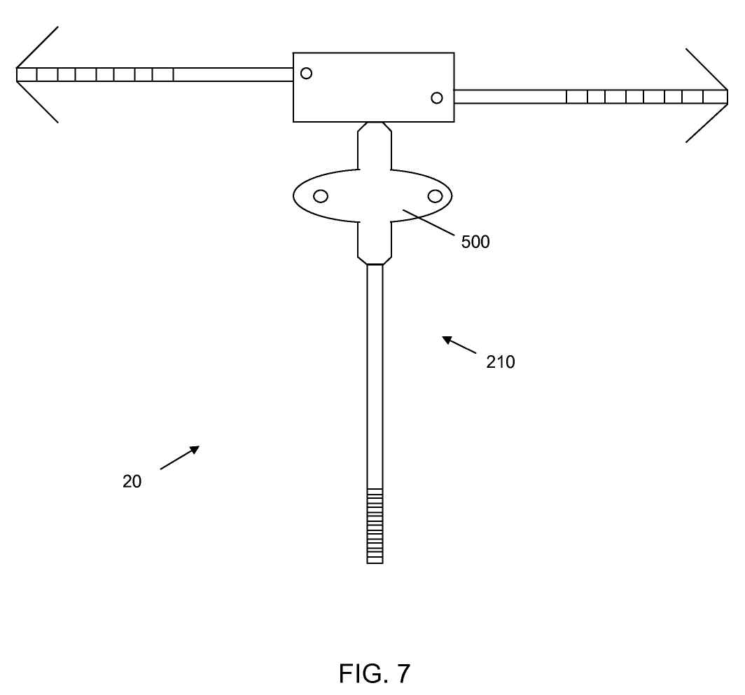

In the embodiment depicted in FIG. 7, an unattached anchor 500 (e.g., an anchor that may be placed on the extension just prior to or during the implant procedure), such as the depicted wing-shaped suture loop anchor, may be disposed about the proximal portion 210 of the extension 20. One or more unattached anchor 500 may be employed and may be employed in addition to or in alternative to an anchor 460 attached to the connector 24, as depicted in FIG. 6.

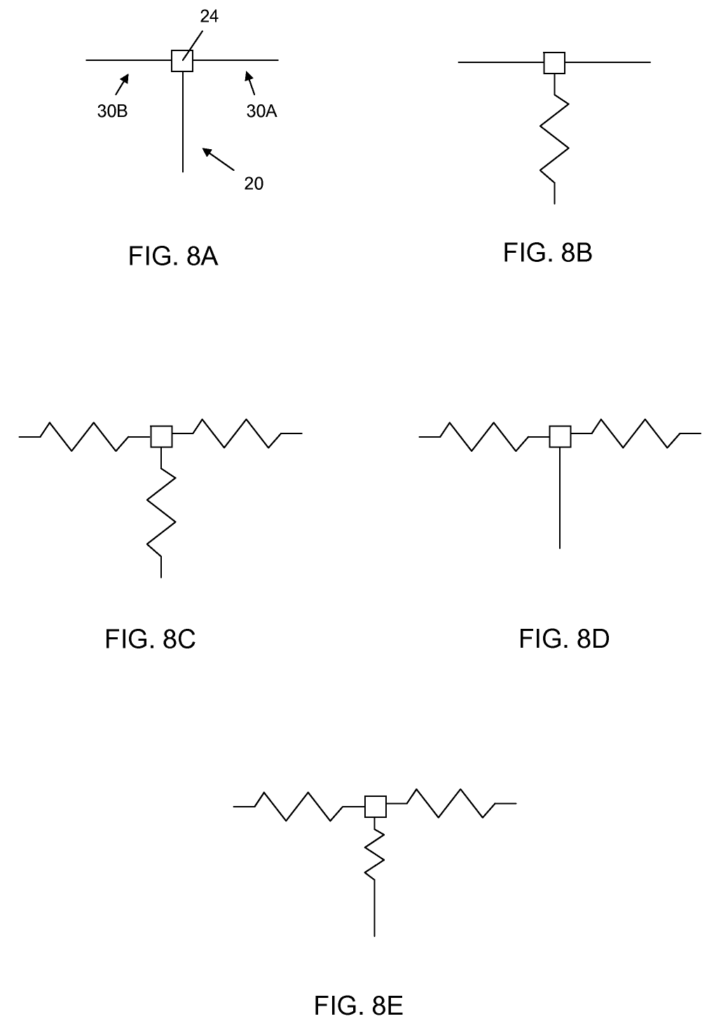

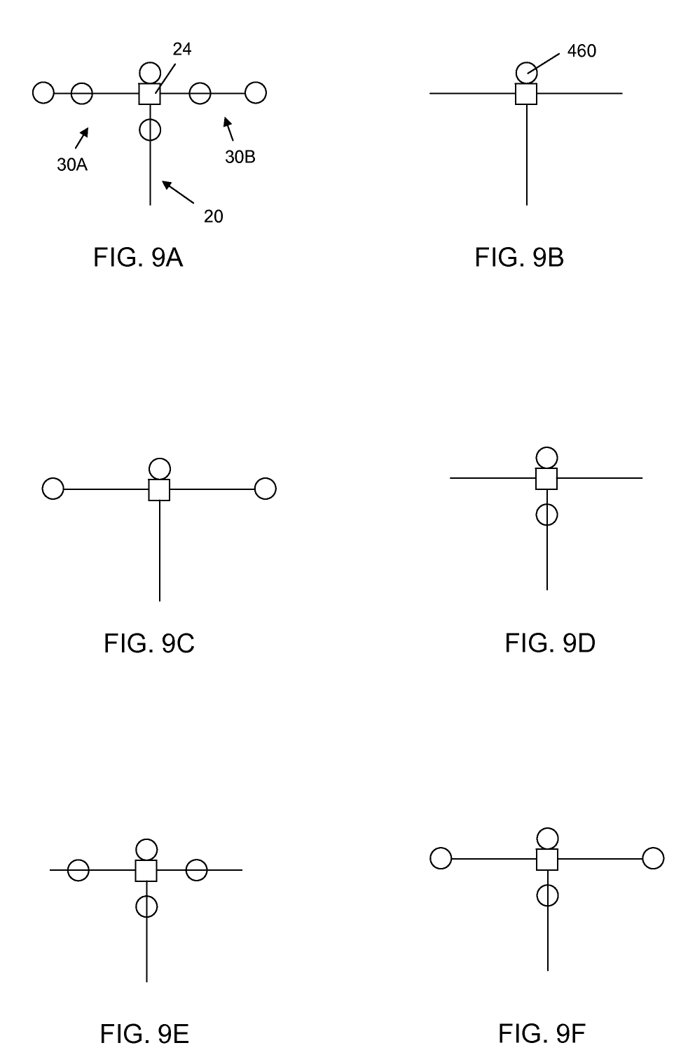

Referring now to FIGS. 8-9, various representative configurations of lead extensions 20 and associated leads 30A, 30B are shown. While T-shaped configurations are depicted, it will be understood that such configurations are readily applicable to Y- or other shaped configurations. In the embodiments depicted in FIGS. 8-9, the extensions 20 include a proximal portion containing contacts (not shown) and a distal connector 24 with associated leads 30A, 30B received by the connector 24. The squiggly lines depicted in FIGS. 8B-E represent extensibility of the proximal portion of the lead extension or of a lead along the squiggly portion. Extensibility may include a sigma shaped section, loops (that may be introduced during implantation or otherwise), or may otherwise be configured to be extensible. As shown in FIGS. 9A-F, in which circles represent anchors 460 that may be attached or unattached, an extension 20 or lead 30A, 30B may include one or more anchor at nearly any location. It will be understood that possible combinations of the configurations shown in FIGS. 8-9 are contemplated, as are combinations of other figured depicted and discussed herein.

Thus, embodiments of LEAD EXTENSION HAVING CONNECTOR CONFIGURED TO RECEIVE TWO LEADS are disclosed. One skilled in the art will appreciate that the leads, extensions, connectors, devices such as signal generators, systems and methods described herein can be practiced with embodiments other than those disclosed. The disclosed embodiments are presented for purposes of illustration and not limitation.

1. A method for applying electrical signals to a left occipital nerve and a right occipital nerve of a subject, the method comprising:
applying a first electrical signal to the left occipital nerve via an electrode of a first lead;
applying a second electrical signal to the right occipital nerve via a second electrode of a second lead;
wherein applying the first and second electrical signal comprises (i) generating the first electrical signal from an electrical signal generator implanted in the subject and transmitting the signal to the electrode of the first lead via a lead extension operably coupled to the first lead, and (ii) generating the second electrical signal from the electrical signal generator and transmitting the signal to the electrode of the second lead via the lead extension, wherein the lead extension is operably coupled to the second lead,
wherein the first and second signals are the same or different.

2. A method according to claim 1,
wherein the lead extension has
a proximal portion that includes first and second contacts and
a distal connector portion having a body housing
(i) a first lead receptacle having an internal contact electrically coupled to the first proximal contact, and
(ii) a second lead receptacle having an internal contact electrically coupled to the second proximal contact,
wherein the first lead comprises
a proximal portion having a contact and
a distal portion comprising the electrode, the contact being electrically coupled to the first electrode,
wherein the second lead comprises
a proximal portion having a contact and
a distal portion comprising the electrode, the contact being electrically coupled to the second electrode,
wherein the method further comprises electrically coupling the first lead to the extension by electrically coupling the contact of the first lead to the internal contact of the first receptacle and electrically coupling the second lead to the extension by electrically coupling the contact of the second lead to the internal contact of the second receptacle.

3. A method according to claim 2, further comprising tunneling the proximal portion of the lead extension from a location of the subject nearer the left and right occipital nerves to a location at which the signal generator is implanted or is to be implanted.

4. A method according to claim 2, further comprising anchoring the connector of the extension to tissue of the subject.

5. A method according to claim 2, further comprising anchoring a portion of the lead extension in proximity to the connector to tissue of the subject.

6. A lead extension comprising:
a proximal portion having first and second contacts; and
a connector having a body housing first and second lead receptacles, the first lead receptacle having an internal contact electrically coupled to the first proximal contact, the second lead receptacle having an internal contact electrically coupled to the second proximal contact,
wherein the first lead receptacle extends into the body from a first opening formed in the body, and the second lead receptacle extends into the body from a second opening formed in the body.

7. A lead extension according to claim 6, wherein the first opening in the body of the connector is formed in a face of the body, and the second opening is formed in a generally opposing face of the connector.

8. A lead extension according to claim 7, wherein the proximal portion enters the connector at an entry region, wherein geometric center of the first opening, the second opening and the entry region are coplanar, and wherein the first and second lead receptacles extend into the body of the connector substantially perpendicular to a line running in the plane through the geometric center of the entry region.

9. A lead extension according to claim 6, wherein the proximal portion enters the connector at an entry region, wherein geometric center of the first opening, the second opening and the entry region are coplanar, and wherein the geometrically centered lines running in the plane extending through the first and second lead receptacles intersect with line running in the plane through the geometric center of the entry region such that an angle of intersection between the geometric center of the entry region and the geometric center of the first opening is greater than 90 degrees and less than 180 degrees and the angle between the geometric center of the entry region and the geometric center of the second opening is greater than 90 degrees and less than 180 degrees.

10. A lead extension according to claim 9, wherein the angle between the geometric center of the entry region and the geometric center of the first opening is between about 110 degrees and about 160 degrees, and wherein the angle between the geometric center of the entry region and the geometric center of the second opening is between about 110 degrees and about 160 degrees.

11. A lead extension according to claim 6, wherein the connector occupies a volume of less than about 10 cubic centimeters.

12. A lead extension according to claim 6, wherein the connector occupies a volume of less than about 5 cubic centimeters.

13. A system comprising:
a lead extension comprising:
a proximal portion having first and second contacts; and
a connector having a body housing first and second lead receptacles, the first lead receptacle having an internal contact electrically coupled to the first proximal contact, the second lead receptacle having an internal contact electrically coupled to the second proximal contact,
wherein the first lead receptacle extends into the body from a first opening formed in the body, and the second lead receptacle extends into the body from a second opening formed in the body; and
an active implantable medical device operably couplable to the lead extension.

14. A system according to claim 13, wherein the active device is an implantable signal generator.

15. A system according to claim 13, further comprising first and second leads operably couplable to the extension.
